(19) United States
(12) Patent Application Publication
(10) Pub. No.: US 2022/0068443 A1
(43) Pub. Date: Mar. 3, 2022

(54) Systems and Methods for Identifying Candidates for Clinical Trials

(71) Applicant: BEKHealth Corporation, Kent, CT (US)

(72) Inventors: Joshua Fuller Ransom, Wayland, MA (US); Jason Baumgartner, Kent, CT (US)

(73) Assignee: BEKHealth Corporation, Kent, CT (US)

(21) Appl. No.: 17/461,286

(22) Filed: Aug. 30, 2021

Related U.S. Application Data
(60) Provisional application No. 63/072,326, filed on Aug. 31, 2020.

(51) Int. Cl. G16H 10/20; G16H 10/60

(52) U.S. Cl. CPC G16H 10/20; G16H 10/60

(57) ABSTRACT

The present disclosure includes systems and methods for determining candidates for clinical trials from unstructured clinical trial protocols associated with the clinical trial and medical records of patients based on machine learning, natural language processing or both. The systems and methods of the present disclosure can extract tokens from unstructured clinical trial protocols based on Natural Language Processing (NLP) and determine clinical trial criteria. The systems and methods of the present disclosure can determine clinical indications from the medical data associated with the patients using natural language processing and determine whether the clinical indications match the clinical trial criteria and determine a probability that the patients meet the clinical trial criteria based on a crosswalk matching and determine candidates for clinical trial from the patients based on the determined probability.

### CROSS REFERENCE TO RELATED APPLICATIONS

This application claims priority to U.S. Provisional Patent Application No. 63/072,326 filed on Aug. 31, 2020 and entitled Systems and Methods for Identifying Candidates for Clinical Trials, the entire contents which hereby incorporated by reference.

### FIELD OF THE DISCLOSURE

The present disclosure generally relates to the field of determining candidates for clinical trials using for example, natural language processing, artificial intelligence and/or machine learning.

### BACKGROUND OF THE DISCLOSURE

Clinical trial protocols for determining candidates for clinical trials are technical documents that do not follow a consistent style and assume the reader is knowledgeable about the technical details and can discern the intent based on his or her knowledge. For example, the writer can assume the reader will understand the intent and requirements of the clinical trial consistent with that of the researcher who drafted the document. This can result in clinical trial sites manually determining the inclusion and exclusion criteria using different decision-making processes while choosing the candidates for the clinical trial. For example, the research staff at a clinical trial site may review years and often decades of medical records to confirm the presence or absence of hundreds of medical terms in order to match a patient to the clinical trial inclusion and exclusion criteria. This process is time consuming and often not consistent across clinical trial sites due to the unstructured data being analyzed and the volume of patient records that must be manually reviewed.

Therefore, there is a need for systems and methods that can efficiently and accurately extract the clinical trial criteria from clinical trial protocols and extract information from the medical records of patients to identify and recruit suitable candidates for clinical trials that match the clinical trial criteria.

### SUMMARY OF THE DISCLOSURE

Embodiments of the present disclosure include systems and methods for determining candidates for clinical trials from unstructured clinical trial protocols associated with the clinical trial and medical records of patients based on Artificial Intelligence (AI), Machine Learning (ML), Natural Language Processing (NLP) or any combination thereof. The systems and methods of the present disclosure can extract protocol tokens from clinical trial protocols based on NLP and determine clinical trial criteria. The clinical trial criteria can include clinical trial inclusion criteria, clinical trial exclusion criteria or both. The clinical trial protocol can be unstructured and written in varying styles of the principal researcher that varies between different researchers and can assume the reader is knowledgeable about the technical details and can discern the intent of the clinical trial protocol based on his or her clinical training. The systems and methods of the present disclosure can determine clinical indications from the medical data associated with the patients using NLP and determine whether the clinical indications match the clinical trial criteria and determine a probability that the patients meet the clinical trial criteria based on a crosswalk matching and determine candidates for clinical trial from the patients based on the determined probability.

In exemplary embodiments, the system and methods can determinate candidates for clinical trials. The system and methods can receive a clinical trial protocol associated with a clinical trial, extract protocol tokens from the clinical trial protocol based on NLP, determine a plurality of clinical trial criteria based on the extracted protocol tokens, receive a plurality of patient medical records associated with a plurality of patients, extract patient tokens from the plurality of patient medical records based on NLP, determine clinical indications of the plurality of patients based on the extracted patient tokens, determine a probability that each of the plurality of patients meet the clinical trial criteria based on a crosswalk matching algorithm, and determine a plurality of clinical trial candidates from the plurality of patients based on the determined probability. In exemplary embodiments, the crosswalk matching algorithm can be based on at least one of: deterministic exact matches between strings, partial-string fuzzy matching, predictive modeling, machine learning, autoencoders, transformers, or any combination thereof.

In exemplary embodiments, the system and methods can determine protected patient information of the candidates for clinical trials based on protected health information (PHI), and output protected patient information to an approved user. In exemplary embodiments, the PHI is based on at least one of: patient demographics, disease diagnoses, medication exposures, medical device exposures, surgeries, medical procedures, lab tests, vital signs, clinical observations, visits to healthcare providers, radiological imaging, imaging reports, pathology images, pathology reports or any combination thereof. In exemplary embodiments, the clinical trial protocol is unstructured data. In exemplary embodiments, the patient medical records are unstructured data.

In exemplary embodiments, the probability that each of the plurality of patients meet the clinical trial criteria is based on at least one of: a patient's interest in clinical research, propensity to consent to participate in a clinical trial, likelihood of adhering to the trial protocol, likelihood of developing adverse events to the investigational medication, or likelihood of experiencing the clinical outcome of interest that the clinical trial is investigating.

In exemplary embodiment, non-transitory computer readable medium storing instructions executable by a processing device, wherein execution of the instructions causes the processing device to implement a method for determining candidates for clinical trials. The system can receive clinical trial protocol associated with a clinical trial, extract tokens from the clinical trial protocol, determine a plurality of clinical trial criteria based on the extracted tokens, receive a plurality of patient medical records associated with a plurality of patients, extract tokens from the plurality of patient medical records, determine clinical properties of the plurality of patients based on the extracted tokens, determine a probability that each of the plurality of patients meet the clinical trial inclusion criteria based on a crosswalk matching algorithm, and determine a plurality of clinical trial candidates from the plurality of patients based on the determined probability.

Any combination or permutation of embodiments is envisioned. Additional advantageous features, functions and applications of the disclosed assemblies, systems and methods of the present disclosure will be apparent from the description which follows, particularly when read in conjunction with the appended figures. The references, publications and patents listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure. To assist those of ordinary skill in the art in making and using the disclosed assemblies, systems and methods, reference is made to the appended figures, wherein:

FIG. 1 illustrates a block diagram of an exemplary system for determining candidates for clinical trials from clinical trial protocols associated with the clinical trial and medical records of patients based on machine learning, natural language processing or both according to the present disclosure;

FIG. 2 illustrates an exemplary flow chart for determining candidates for clinical trial according to the present disclosure;

FIG. 3 illustrates an exemplary flow chart for determining candidates for clinical trial according to the present disclosure; and

FIG. 4 illustrates an exemplary block diagram of an exemplary computing device for implementing exemplary embodiments of the present disclosure.

### DETAILED DESCRIPTION OF THE DISCLOSURE

The exemplary embodiments disclosed herein are illustrative of methods and related systems for determining candidates for clinical trials from clinical trial protocols associated with the clinical trial and medical records of patients based on AI, ML, NLP or a combination thereof according to the present disclosure. The system and method can extract tokens from clinical trial protocols based on AI, ML, NLP or a combination thereof and determine clinical trial criteria. The system and method can extract patient tokens to extract clinical indications, from patient reports such as patient natural histories, patient treatment/regimen line of therapy, patient risk stratification, patient disease progression, patient discharge notes, laboratory reports, pathology reports, radiology reports, customer relationship management, patient case management, patient care coordination, and precision patient interventions to determine the clinical trial criteria and processes for the clinical trial. The system and method can determine the probability of meeting the clinical trial criteria based on a crosswalk matching algorithm. The system and method can determine candidates for the clinical trial based on the determined probability.

Details disclosed herein with reference to exemplary systems/assemblies and associated processes/techniques of assembly and use are not to be interpreted as limiting, but merely as the basis for teaching one skilled in the art how to make and use the advantageous assemblies, systems and methods of the present disclosure.

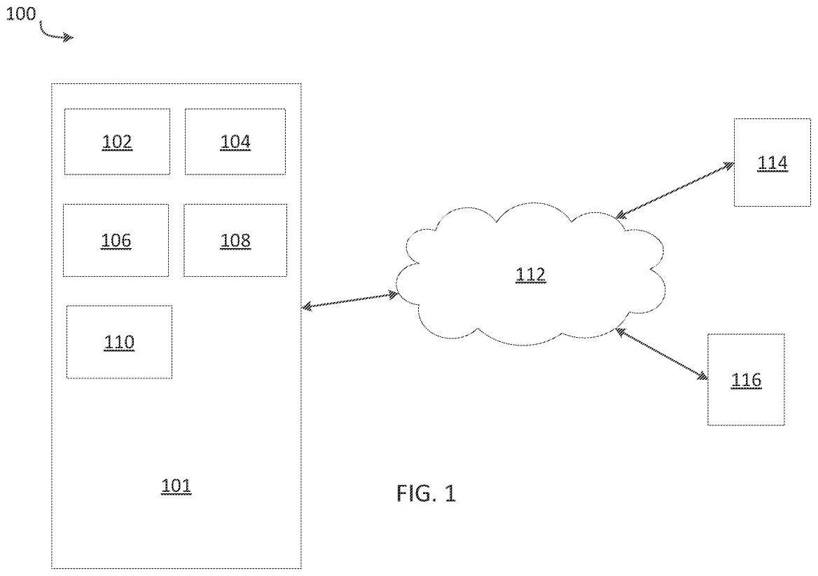

With reference to FIG. 1, an illustration of the system 100 for determining candidates for clinical trials from clinical trial protocols associated with the clinical trial and medical records of patients based on AI, ML, NLP, or a combination thereof according to the present disclosure is provided. The system 100 includes individually scalable sub-systems such as an NLP system 101. The NLP system 101 can include scalable sub-systems such a machine learning model trainer 102 for training the machine learning model 104 and a machine learning interpreter 106 to use the trained machine learning model 104 to interpret a received clinical trial protocol 108. The system 100 can be connected via a network 112 to receive inputs.

In an exemplary embodiment, the machine learning model trainer 102 can receive a database that includes a plurality of clinical trial protocols of previous trials, patients that were considered for the clinical trial, the patient records for the patients, the candidates that were chosen for the clinical trial and the outcome of the clinical trial for each candidate. The machine learning model trainer 102 can normalize the unstructured data from the plurality of clinical trial protocols of previous trials to extract tokens. For example, the tokens can include words or phrases from the clinical trial protocols that have been normalized. The machine learning model trainer 102 can for example use stemming, lemmatization, canonization, removal of stop words, or a combination thereof to extract tokens. In an example, the extracted token can include the medical terminology, the processes of the clinical trial and the like. The machine learning model trainer 102 can similarly tokenize the patient records from the previous trials to determine the clinical record of the patients and the candidates that were chosen for the clinical trial.

In an example, the machine learning model trainer 102 can use an NLP algorithm to train the machine learning model 104 to determine clinical trial criteria. The machine learning model trainer 102 can also train the machine learning 104 to determine patients that match the clinical trial criteria to determine patients that match the clinical trial criteria based on the records of the patients. In exemplary embodiments, the machine learning model trainer 102 can generate machine learning models for different clinical trial criteria such as clinical trial terminology, medical codes, and other patient characteristics. Similarly, the machine learning model trainer 102 can generate machine learning models for patient medical records such as relational clinical terminology, medical dictionary codes and other patient characteristics.

In an exemplary embodiment, the system 100 can receive the clinical trial protocol 108 via a text input. For example, the system can receive the clinical trial protocols 108 from the principal researcher designing the clinical trial in text format. The clinical trial protocols 108 can include phrases that describe clinical trial criteria such as clinical trial inclusion criteria and clinical trial exclusion criteria. The clinical trial criteria can describe the requirements for the clinical trial such as the patient natural history, patient clinical history, the patient treatment regimen, the patient risk stratification, the patient disease progression, the patient case management, the patient care coordination, the patient interventions, the processes for selecting candidate for the clinical trial and the like. For example, the clinical trial protocols 108 can describe the patient characteristics of ideal candidates for the clinical trial in addition to the clinical trial criteria such as a patient's interest in clinical research, propensity to consent to participate in a clinical trial, likelihood of adhering to the trial protocol, likelihood of developing adverse events to the investigational medication, or likelihood of experiencing the clinical outcome of interest that the clinical trial is investigating. The machine learning interpreter 106 can use the machine learning model 104 to tokenize the clinical trial protocol 108 and determine clinical trial criteria. For example, the machine learning model interpreter 106 can parse the description of the clinical trial protocol 108 for the associated medical terminology and/or associated parts of speech such as threshold modifiers, lab units, treatment dosage, treatment routes of administration, disease severity, negation detection and the like. In an exemplary embodiment, the system 100 can crosswalk the medical terminology against medical dictionary codes to normalize the terminology. In an exemplary embodiment, the system 100 can crosswalk the medical terminology against lookup tables to normalize and standardize at least one of: treatment dosage equivalents, lab units, time between medical events, frequency of medical event occurrence, or any combination thereof. In an exemplary embodiment, the machine learning model interpreter 106 can use the machine learning models of clinical trial criteria in stacks to determine the clinical trial criteria. The system 100 can determine the patient tokens from sources such as patient natural histories, patient treatment/regimen line of therapy, patient risk stratification, patient disease progression, patient discharge notes, laboratory reports, pathology reports, radiology reports, customer relationship management, patient case management, patient care coordination, and precision patient interventions that correspond to the protocol tokens from the clinical trial protocol 108. In an exemplary embodiment, the machine learning interpreter 106 can use the machine learning models of patient medical records in stacks to determine the patient tokens.

The system 100 can receive patient medical records 110. For example, the system 100 can receive the patient medical records 110 from a candidate data repository. The patient medical records 110 can be a mixture of structured data and unstructured data. For example, the patient medical records 110 can include codes describing diagnosis, treatments and the like for the patients. The machine learning interpreter 106 can use the machine learning model 104 to tokenize the patient medical records 110 and to determine clinical indications of the plurality of patients based on the extracted patient tokens. The system 100 can determine a probability that each of the plurality of patients meet the clinical trial criteria based on the crosstalk matching algorithm. For example, the machine learning interpreter 106 can determine whether each of the plurality of patient records has the medical terminology, cross-walked medical dictionary codes or both from the clinical trial protocol 108.

The system 100 can determine the plurality of clinical trial candidates from the patients based on the determined probability. In examples, the system 100 can determine the clinical trial candidates based on patient characteristics that make them good clinical trial participants in addition to the direct criteria matching. Examples of patient characteristics can include a patient's interest in clinical research, propensity to consent to participate in a clinical trial, likelihood of adhering to the trial protocol, likelihood of developing adverse events to the investigational medication, or likelihood of experiencing the clinical outcome of interest that the clinical trial is investigating. In an exemplary embodiment, the system 100 can determine the risk/propensity scores for each inclusion/exclusion criteria for each patient to determine if patients with imperfect matches on one or more inclusion/exclusion criteria may still be evaluated as candidates for the clinical trial.

In an exemplary embodiment, the system 100 can determine based on the machine learning model 104 to calculate probabilities of patients matching one or more of the relational clinical terminology, medical dictionary codes, and/or other patient characteristics at a future date.

In an exemplary embodiment, the patient medical records 110 can be anonymized to protect the identity of the patients. The system 100 can determine the protected patient information of the candidates for clinical trial inclusion based on the protected health information that corresponds to the patient medical records 110. The system 100 can output the patient information to an approved user 116. For example, the system 100 can output the candidates matching all the clinical protocol criteria. In another example, the system 100 can output the candidates that are the closest match for the clinical protocol criteria. In an exemplary embodiment, the system 100 can output list of matched patients, their visits, and healthcare providers are then presented back to the clinical research staff in order to facilitate recruiting and enrolling the patient into the clinical trial.

Examples of machine learning algorithms that can be implemented via the system 100, can include, but are not limited to Linear Regression, Logical Regression, Decision Tree, Support Vector Machine, Naïve Bayes, k-Nearest Neighbors, k-Means, Random Forest, Dimensionality Reduction algorithms (such as GBM, XGBoost, LightGBM and CatBoost), Deep Learning Neural Network algorithms (such as Perceptron, Recurrent Neural Network, Long/Short Term Memory, Auto-Encoder, Denoising Auto-Encoder, Deep Convolutional Inverse Graphics Network, Markov Chain, Deep Convolutional Network, Deconvolutional Network, Deep Bidirectional Transformers).

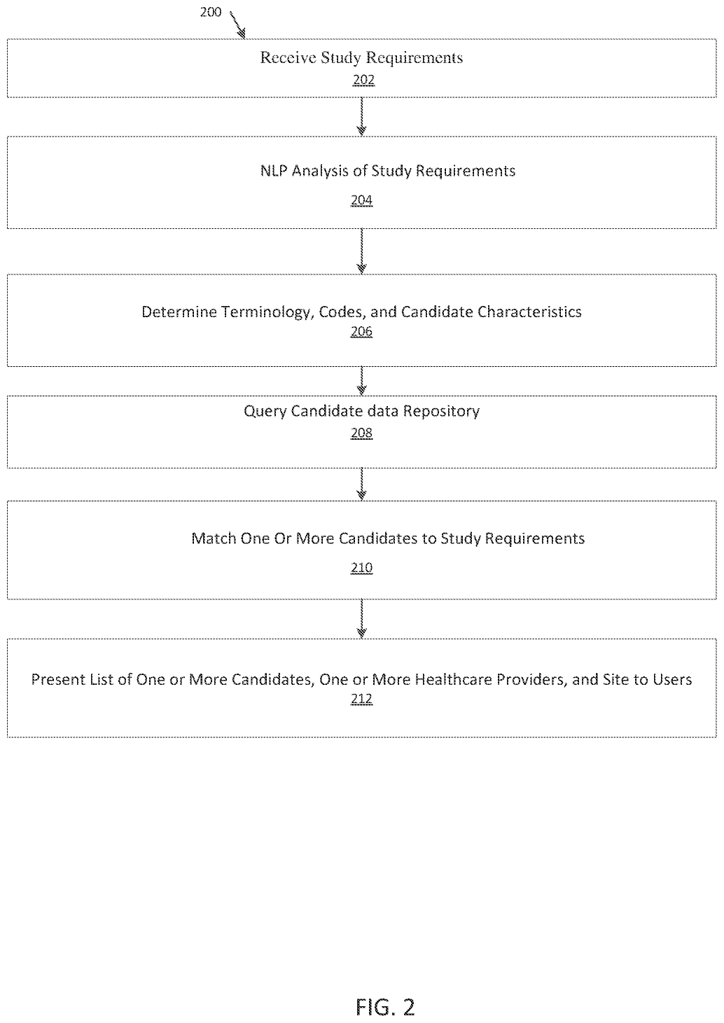

With reference to FIG. 2, the system 100 can use the work flow 200 illustrated in a flow chart to determine candidates for clinical trial. The operations 202 to 212 describe the process of determining candidates for the clinical trial in accordance with an embodiment described herein. In operation 202, the system 100 can receive study requirements such as the clinical trial protocol 108. For example, the system 100 can receive study requirements from the principal investigator in text format. In operation 204, the system 100 can perform natural language processing on the study requirements such as the clinical trial protocol 108. For example, the system 100 can use NLP algorithms such as word2vec, term frequency—inverse document frequency, or pre-trained transformers, to determine the clinical trial criteria. The clinical trial criteria can include inclusion criteria or exclusion criteria. In operation 206, the system 100 can determine the terminology, codes and candidate characteristic based on the clinical trial criteria. In operation 208, the system 100 can query the candidate data repository to compare the clinical trial criteria with the patient records of the candidates to determine a probability that the patient matches the clinical trial criteria. In operation 210, the system 100 can match one or more candidates that meet the clinical trial criteria or study requirements based on the determined probability. In operation 212, the system 100 can present a list of one or more candidates, one or more healthcare providers and location, to authorized users. For example, the system 100 can determine the healthcare provider of the candidate that meets the study requirements from the candidate data repository.

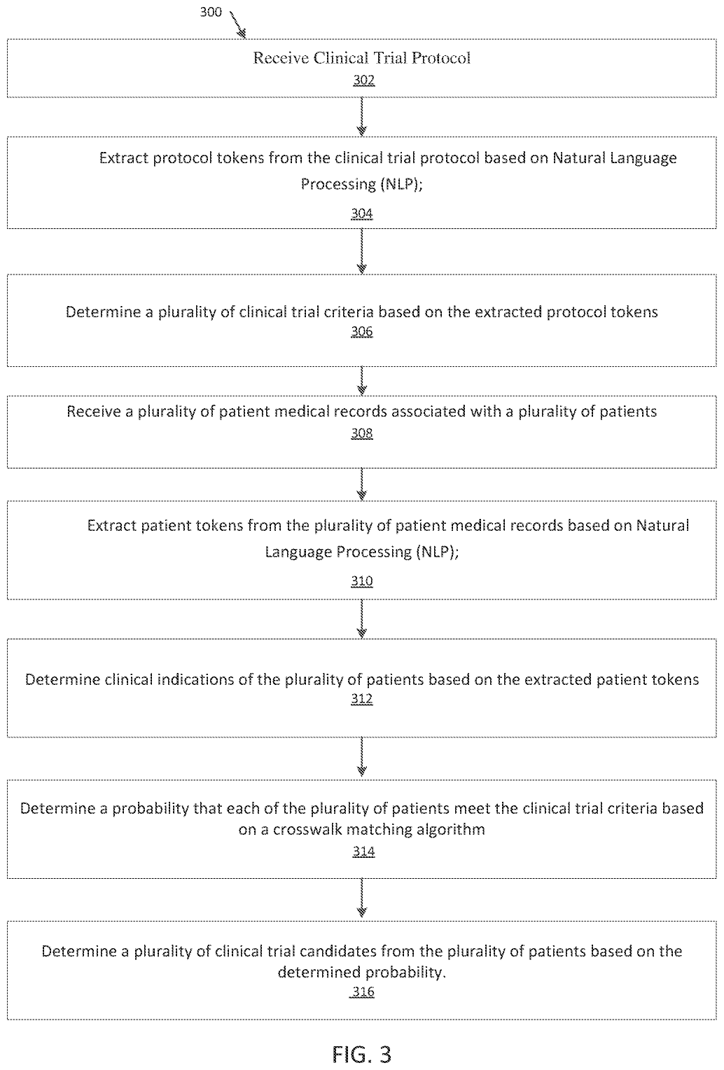

With reference to FIG. 3, the system 100 can use the work flow 300 illustrated in a flowchart to determine candidates for clinical trials in accordance with an embodiment described herein. The operations 302 to 316 describe the process of determining candidates for the clinical trial in accordance with an embodiment described herein. In operation 302, the system 100 can receive clinical trial protocol 108.

In operation 304, the system 100 can determine protocol tokens from the clinical trial protocol 108 based on NLP. For example, the system 100 can tokenize the clinical trial protocol by stemming, canonization and removal of stop words.

In operation 306, the system 100 can determine a plurality of clinical trial criteria based on the extracted protocol tokens. For example, the system 100 can determine the clinical trial criteria based on the machine learning model. In operation 308, the system 100 can receive a plurality of patient medical records 110 associated with a plurality of patients. The patient medical records 110 can be a mixture of structured data and unstructured data. In operation 310, the system 100 can extract patient tokens from the plurality of patient medical records based on NLP. In operation 312, the system 100 can determine clinical indications of the plurality of patients based on the extracted patient tokens. For example, the system 100 can use the machine learning model 104 to determine the clinical indications of the patients from the patient medical records 110. In operation 314, the system 100 can determine a probability that each of the plurality of patients meet the clinical trial criteria based on a crosswalk matching algorithm. For example, the system 100 can determine the patient tokens such as patient natural histories, patient treatment/regimen line of therapy, patient risk stratification, patient disease progression, patient discharge notes, laboratory reports, pathology reports, radiology reports, customer relationship management, patient case management, patient care coordination, and precision patient interventions that correspond to the protocol tokens from the clinical trial protocol 108. In operation 316, the system 100 can determine a plurality of clinical trial candidates from the plurality of patients based on the determined probability.

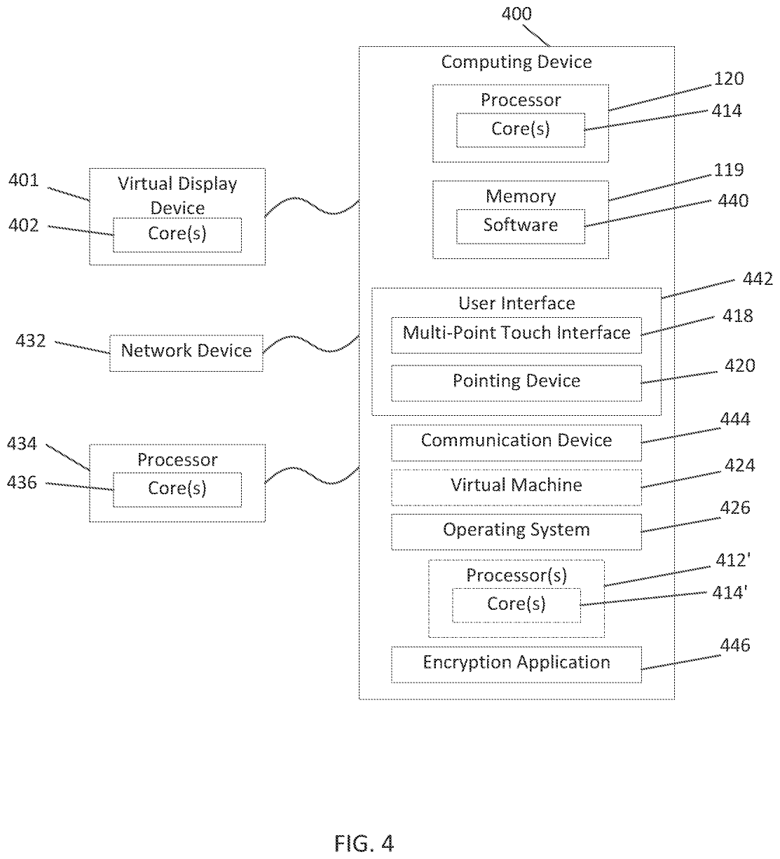

With reference to FIG. 4, a block diagram of an example computing device for implementing exemplary embodiments of the present disclosure is illustrated. An exemplary embodiment for determining candidates for clinical trials can be implemented by a computing device 400. The computing device 400 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 119 included in the computing device 400 may store computer-readable and computer-executable instructions or software (e.g., applications) for implementing exemplary operations of the computing device 400. The computing device 400 also includes configurable and/or programmable processor 434 and associated core(s) 436 and, optionally, one or more additional configurable and/or programmable processor(s) 412′ and associated core(s) 414′ (for example, in the case of computer systems having multiple processors/cores), for executing computer-readable and computer-executable instructions or software stored in the memory 406 and other programs for implementing exemplary embodiments of the present disclosure. Processor 402 and processor(s) 402′ may each be a single core processor or multiple core (404 and 404′) processor. Either or both of processor 402 and processor(s) 402′ may be configured to execute one or more of the instructions described in connection with computing device 400. Processor 402 and processor(s) 402′ may each be a central processing unit (CPU), graphical processing unit (GPU), tensor processing unit (TPU), or any combination thereof.

Virtualization may be employed in the computing device 400 so that infrastructure and resources in the computing device 400 may be shared dynamically. A virtual machine 412 may be provided to handle a process running on multiple processors so that the process appears to be using only one computing resource rather than multiple computing resources. Multiple virtual machines may also be used with one processor.

Memory 406 may include a computer system memory or random-access memory, such as DRAM, SRAM, EDO RAM, and the like. Memory 406 may include other types of memory as well, or combinations thereof. A user may interact with the computing device 400 through a visual display device 414, such as a computer monitor, which may display one or more graphical user interfaces 416, multi-touch interface 420, and a pointing device 418. The computing device 1700 may also include one or more storage devices 426, such as a hard-drive, CD-ROM, or other computer-readable media, for storing data and computer-readable instructions and/or software that implement exemplary embodiments of the present disclosure (e.g., applications). For example, exemplary storage device 426 can include one or more databases 428 for storing information regarding the physical objects. The databases 428 may be updated manually or automatically at any suitable time to add, delete, and/or update one or more data items in the databases.

The computing device 400 can include a network interface 408 configured to interface via one or more network devices 424 with one or more networks, for example, Local Area Network (LAN), Wide Area Network (WAN) or the internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. In exemplary embodiments, the computing system can include one or more antennas 422 to facilitate wireless communication (e.g., via the network interface) between the computing device 400 and a network and/or between the computing device 400 and other computing devices. The network interface 408 may include a built-in network adapter, network interface card, PCMCIA network card, card bus network adapter, wireless network adapter, USB network adapter, modem or any other device suitable for interfacing the computing device 400 to any type of network capable of communication and performing the operations described herein.

The computing device 400 may run any operating system 410, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, or any other operating system capable of running on the computing device 400 and performing the operations described herein. In exemplary embodiments, the operating system 410 may be run in native mode or emulated mode. In an exemplary embodiment, the operating system 410 may be run on one or more cloud machine instances.

The computing device 400 can include an encryption application 446 that encrypts PHI when stored or when PHI is transmitted between parts of the system to prevent unauthorized access. For example, the computing device 400 can have virtualized sub-systems that use the encryption application 446 to encrypt PHI when transmitting information between the subsystems.

Exemplary flowcharts are provided herein for illustrative purposes and is a non-limiting example of a method. One of ordinary skill in the art will recognize that exemplary methods may include more or fewer steps than those illustrated in the exemplary flowcharts.

We claim:

1. A system for determining candidates for clinical trials, the system comprising:
at least one processor operatively connected to a memory containing instructions, that when executed cause the at least one processor to:
receive a clinical trial protocol associated with a clinical trial;
extract protocol tokens from the clinical trial protocol based on Natural Language Processing (NLP);
determine a plurality of clinical trial criteria based on the extracted protocol tokens;
receive a plurality of patient medical records associated with a plurality of patients;
extract patient tokens from the plurality of patient medical records based on NLP;
determine clinical indications of the plurality of patients based on the extracted patient tokens;
determine a probability that each of the plurality of patients meet the clinical trial criteria based on a crosswalk matching algorithm; and
determine a plurality of clinical trial candidates from the plurality of patients based on the determined probability.

2. The system in claim 1, wherein the at least one processor is configured to:
determine protected patient information of the candidates for clinical trials based on protected health information; and
output protected patient information to an approved user.

3. The system in claim 1, wherein the probability that each of the plurality of patients meet the clinical trial criteria is based on at least one of: a patient's interest in clinical research, propensity to consent to participate in a clinical trial, likelihood of adhering to the trial protocol, likelihood of developing adverse events to the investigational medication, or likelihood of experiencing the clinical outcome of interest that the clinical trial is investigating.

4. The system in claim 1 wherein, the clinical trial protocol is unstructured data.

5. The system in claim 1, wherein the clinical trial protocol is a combination of structured data and unstructured data.

6. The system in claim 1, wherein the patient medical records are unstructured data.

7. The system in claim 1, wherein the patient medical records are a combination of structured data and unstructured data.

8. The system in claim 1, wherein the clinical trial criteria include clinical trial exclusion criteria, clinical trial exclusion criteria or both.

9. The system in claim 1, wherein the probability that each of the plurality of patients meet the clinical trial criteria is based on patient characteristics in the future.

10. A method for determining candidates for clinical trials, the method comprising:
receiving, via a Natural Language Processing (NLP) system, clinical trial protocol associated with a clinical trial;
extracting, via the NLP system, tokens from the clinical trial protocol;
determining, via the NLP system, a plurality of clinical trial criteria based on the extracted tokens;
receiving, via the NLP system, a plurality of patient medical records associated with a plurality of patients;
extracting, via the NLP system, tokens from the plurality of patient medical records;
determining, via the NLP system, clinical properties of the plurality of patients based on the extracted tokens;
determining, via the NLP system, a probability that each of the plurality of patients meet the clinical trial inclusion based on a crosswalk matching algorithm; and
determine, via the NLP system, a plurality of clinical trial candidates from the plurality of patients based on the determined probability.

11. The method in claim 10, further comprising:
determining, via the NLP system, protected patient information of the candidates for clinical trials based on protected health information; and
outputting, via the NLP system, protected health information to an approved user.

12. The method in claim 10, wherein the clinical trial protocol is unstructured data.

13. The method in claim 10, wherein the patient medical records are unstructured data.

14. The method in claim 10, wherein the probability that each of the plurality of patients meet the clinical trial criteria is based on at least one of: a patient's interest in clinical research, propensity to consent to participate in a clinical trial, likelihood of adhering to the trial protocol, likelihood of developing adverse events to the investigational medication, or likelihood of experiencing the clinical outcome of interest that the clinical trial is investigating.

15. A non-transitory computer readable medium storing instructions executable by a processing device, wherein execution of the instructions causes the processing device to implement a method for determining candidates for clinical trials, the method comprising:
receiving, via a Natural Language Processing (NLP) system, clinical trial protocol associated with a clinical trial;
extracting, via the NLP system, protocol tokens from the clinical trial protocol;
determining, via the NLP system, a plurality of clinical trial criteria based on the extracted protocol tokens;
receiving, via the NLP system, a plurality of patient medical records associated with a plurality of patients;
extracting, via the NLP system, patient tokens from the plurality of patient medical records;
determining, via the NLP system, clinical properties of the plurality of patients based on the extracted patient tokens;
determining, via the NLP system, a probability that each of the plurality of patients meet the clinical trial criteria based on a crosswalk matching algorithm; and
determine, via the NLP system, a plurality of clinical trial candidates from the plurality of patients based on the determined probability.
